(19) United States
(12) Patent Application Publication
(10) Pub. No.: US 2026/0069606 A1
(43) Pub. Date: Mar. 12, 2026

(54) Oxidative Stress and Diabetes Treatment with Cobinamide

(71) Applicant: The Regents of the University of California, Oakland, CA (US)

(72) Inventors: Gerard Boss, La Jolla, CA (US); Renate B. Pilz, La Jolla, CA (US); Adriano Chan, San Diego, CA (US); Sameh S. Ali, Giza (EG); Hema Kalyanaraman, Oakland, CA (US); Darren E. Casteel, Oakland, CA (US); Shyamsundar Pal China, Oakland, CA (US); John Tat, Oakland, CA (US); Stephen Chang, Oakland, CA (US)

(21) Appl. No.: 19/108,605

(22) PCT Filed: Aug. 31, 2023
(86) PCT No.: PCT/US2023/031660

§ 371 Date: Mar. 4, 2025

Related U.S. Application Data
(60) Provisional application No. 63/404,097, filed on Sep. 6, 2022.

(51) Int. Cl. A61K 31/555; A61P 39/06

(52) U.S. Cl. CPC A61K 31/555; A61P 39/06

(57) ABSTRACT

Compositions and methods for treating oxidative stress, and conditions where oxidative stress contributes to conditions such as diabetes, in a subject by administering an effective amount of cobinamide. Oxidative stress can be caused by, for example, increased levels of reactive oxygen species (ROS) and reactive nitrogen species (RNS) in the patient. Compositions and methods for treating oxidative stress that contributes to diabetes-associated conditions such as cardiac fibrosis, and to aortic aneurysm, lipid or protein oxidation, or DNA damage, in a subject by administering an effective amount of cobinamide.

### CROSS-REFERENCE TO RELATED APPLICATION

This application claims priority to U.S. Provisional Application No. 63/404,097 filed on Sep. 6, 2022, the entire contents of which are incorporated by reference.

### GOVERNMENT SPONSORSHIP

This invention was made with government support under Grant No. U01 NS058030 awarded by the National Institutes of Health. The government has certain rights in the invention.

### TECHNICAL FIELD

The present invention relates generally to use of compounds to treat oxidative stress.

### BACKGROUND

Cobinamide is a late precursor in cobalamin (vitamin B12) biosynthesis by microorganisms¹. It lacks the dimethylbenzimidazole ligand of cobalamin, which is replaced by water in aqueous solutions (FIGS. 1A-1B). This imparts several important chemical differences between cobinamide and cobalamin. First, cobinamide can bind two ligands instead of only one. Second, the bulky dimethylbenzimidazole ligand of cobalamin reduces the affinity of the cobalt for ligands in the trans position². Third, cobinamide is considerably more water soluble and lacking the relatively labile phosphodiester group is more stable in aqueous solutions than cobalamin^{3,4}. And fourth, relevant to the results reported here, the cobalt in cobinamide is more easily reduced than the cobalt in cobalamin, likely due to the considerably lower electron-donating ability of water compared to dimethylbenzimidazole: the standard potential for the reduction of the cobalt from the +3 oxidation state to the +2 oxidation state in cobalamin is-40 mV compared to +270 m V for the cobalt in cobinamide^{5,6}. As used herein, cobinamide in the +3 oxidation state is abbreviated Cbi(III) and cobinamide in the +2 oxidation state is abbreviated Cbi(II).

It has been shown in numerous in vitro studies in cultured cells and in vivo studies in fruit flies, mice, rabbits, and pigs that cobinamide is an excellent cyanide and hydrogen sulfide antidote, much better than cobalamin, an approved treatment for cyanide poisoning⁷⁻¹¹. Cobinamide and cobalamin both serve as cyanide and sulfide scavengers, but due to cobinamide's higher affinity for ligands, cobinamide is a more efficient scavenger². Cyanide and sulfide inhibit cytochrome c oxidase in complex IV of the mitochondrial electron transport chain, thereby generating superoxide (O₂·⁻) and inducing intracellular oxidative stress^{10,12}. Cobalamin with the cobalt in the +2 oxidation state reacts with O₂·⁻, peroxynitrite (ONOO⁻), and hypochlorous acid, and in both the +3 and +2 oxidation states with hydrogen peroxide (H₂O₂) and the carbonate radical anion (CO₃·⁻)¹³⁻¹⁸. Consistent with these in vitro data, cobalamin functions as an antioxidant in several cell and animal model systems¹⁹⁻²³. It has been shown that cobinamide reduces oxidative stress in sulfide-poisoned mice, suggesting that part of cobinamide's and cobalamin's activity against cyanide and hydrogen sulfide is due to their antioxidant properties¹⁰.

### SUMMARY OF THE INVENTION

Disclosed herein are compositions and methods for treating oxidative stress, and conditions associated with oxidative stress such as diabetes, in a subject in need thereof with an effective amount of cobinamide. Oxidative stress can be caused by, for example, increased levels of reactive oxygen species (ROS) and reactive nitrogen species (RNS) in the patient.

In embodiments, the disclosure provides a method of treating oxidative stress in a subject with diabetes, where the method includes administering to a subject in need thereof an effective amount of cobinamide, a cobinamide derivative, or a salt thereof. In some embodiments, the vascular disease of diabetes is associated with oxidative stress. In some embodiments, the cobalt atom of the cobinamide, cobinamide derivative, or salt thereof independently may be coordinated with one or more ligands. In some embodiments, the cobinamide, cobinamide derivative, or salt thereof is administered orally.

In embodiments, the disclosure provides a method of treating oxidative stress in a subject, where the method includes administering to a subject in need thereof an effective amount of cobinamide, a cobinamide derivative, or a salt thereof. In some embodiments, the oxidative stress contributes to diabetes, cardiac fibrosis, aortic aneurysm, lipid or protein oxidation, or DNA damage.

In embodiments, the disclosure provides a method of treating oxidative stress associated with diabetes in a subject, and in particular vascular disease due to diabetes, where the method includes administering to a subject in need an effective amount of a composition comprising an anti-oxidant to lower reactive oxygen species. In some embodiments, the anti-oxidant is cobinamide, a cobinamide derivative, or a salt thereof.

### BRIEF DESCRIPTION OF THE DRAWINGS

FIGS. 1A-1G. Cobinamide Functions as a Superoxide Dismutase Mimetic. FIGS. 1A-1B. Chemical structures of aquohydroxo-cobinamide (FIG. 1A) and hydroxo-cobalamin (FIG. 1B). FIGS. 1C, 1E. Increasing concentrations of Chi(III) (FIG. 1C) or Chi(II) (FIG. 1E) were incubated in a hypoxanthine-xanthine oxidase-cytochrome c-catalase system for measuring O₂·⁻, with the change in absorbance at 550 nm (ΔAbs) plotted versus time. A no substrate (no hypoxanthine) control was included. FIGS. 1D, 1F. The calculated rate of change of absorbance at 550 nm in the absence of Chi(III) or Chi(II) (Vₒ) divided by the calculated rate in their presence (v) is plotted against the Chi(III) (FIG. 1D) or Chi(II) (FIG. 1F) concentration, respectively. The slope of the fitted line was used to calculate the apparent rate constant according to the following equation: k_{Cbi}=slope×k_{cytochrome c}×[cytochrome c], where k_{cytochrome c}=1.4×10⁶ M⁻¹ s⁻¹ and [cytochrome c]=70 μM⁷⁰. The data shown are the mean of duplicate samples from one experiment, with similar results found in two additional experiments. Dotted lines show 95% confidence intervals. FIG. 1G. A 20 μM solution of Chi(III) in DMSO was scanned from 280 to 600 nm yielding the spectrum shown as a black line. An equimolar amount of potassium oxide (KO₂) dissolved in dimethyl sulfoxide (DMSO) was added and the solution was scanned immediately, yielding the grey line. The inset shows Chi(II) in DMSO generated by ascorbate reduction of Chi(III). The experiment was conducted three times with identical results.

FIGS. 2A-2F. Cobinamide Functions as a Catalase Mimetic and Reacts with Peroxynitrite. FIGS. 1A-1D. Hydrogen peroxide (final concentration 50 μM) was added to solutions containing increasing concentrations of Chi(III) (FIG. 2A) or Chi(II) (FIG. 2C), and the H₂O₂ concentration was measured over time using a H₂O₂-specific electrode. The log of the velocity in the presence of Chi(III) (FIG. 2B) or Chi(II) (v) (FIG. 2D) over the velocity in their absence (Vₒ) is plotted against the log of their concentrations at 20 min over their concentrations at zero time. These plots yield a reaction order with respect to Chi(III) and Cbi(II) of 0.7. The experiments were repeated three times with similar results. Dotted lines show 95% confidence intervals. FIG. 2E. Adding an equimolar amount of peroxynitrite to a 25 μM Cbi(II) solution in 50 mM sodium phosphate, pH 11 immediately changed the UV-visible spectrum to that of Chi(III) (inset shows the spectrum of authentic Cbi(III) at pH 11). The Chi(III) spectra are slightly different from those shown in FIG. 1G because at pH 11, dihydroxo-cobinamide is generated, whereas at pH 7.1, aquohydroxo-cobinamide is the predominant species. The spectra of aquohydroxo- and dihydroxo-cobinamide are known to be different⁶⁵. FIG. 2F. Varying concentrations of Chi(II) from 1 to 10 μM were mixed with 10 μM peroxynitrite in a stopped-flow instrument, and absorption at 315 nm was monitored for 0.5 sec. The results for 5 μM Cbi(II) are shown; red circles are the observed data, and the black line is a two-phase non-linear regression curve generated by Prism 7.04 software. The inset shows a plot of k_{observed} versus the Cbi(II) concentration, yielding an apparent rate constant for the reaction between Cbi(II) and peroxynitrite of 6.34×10⁶ M⁻¹ s⁻¹. The experiment was repeated three times with similar results. Dotted lines show 95% confidence intervals.

FIGS. 3A-3H. Cobinamide Rescues Cells from Oxidative Stress, and Is Superior to Other Antioxidants; Reduction of Cbi(III) by Ascorbate, Cysteine, and Reduced Glutathione. FIGS. 3A-3B. H9c2 cells were incubated for 30 min with vehicle, 2.5 μM cobinamide, or 5 μM rotenone, the latter in the absence or presence of 2.5 μM cobinamide, imisopasem, MnTBAP, or cobalamin; 5 μM MitoSOX was added during the last 10 min. The cells were incubated with 1 mg/ml DAPI for 3 min to counterstain nuclei, and then visualized under a fluorescence microscope. FIG. 3A. Equal-sized representative areas for each condition are shown for MitoSOX staining (red fluorescence) and the same area showing merged MitoSOX and DAPI staining. FIG. 3B. The amount of red fluorescence was quantified by Image J analysis. Data are the mean±SD of three independent experiments; in each experiment two separate equal-sized areas were analyzed containing ˜75 cells per area. FIGS. 3C-3D. H9c2 cells were incubated for 10 min with 10 μM JC-1, washed once with PBS, and then incubated for 30 min with vehicle, 2.5 μM cobinamide, or 5 μM rotenone in the absence or presence of 2.5 μM of the indicated drugs as described in Panels A, B. FIG. 3C. Cells were visualized under a fluorescence microscope and equal-sized representative areas of each condition are shown, with red and green fluorescence shown for the same area. FIG. 3D. The amount of red and green fluorescence was quantified by Image J analysis and the red to green ratio was calculated. The slides were analyzed by an operator who was blinded to the specific conditions. Data are the mean±SD of three independent experiments; in each experiment two separate equal-sized areas were analyzed containing ˜75 cells per area. FIGS. 3E-3F. H9c2 cells were incubated for 30 min in the absence of any addition (control, Con) or with 100 μM H₂O₂ in the absence (vehicle, Veh) or presence of 100 μM of the indicated drugs. The cells were extracted in a SDS-based buffer, proteins were resolved by PAGE, and phospho-JNK (Thr¹⁸³/Tyr¹⁸⁵) (pJNK) was identified by immunoblotting (upper blot). The two isoforms of pJNK have observed molecular weights of 46 and 54 kDa. The blot was stripped and re-probed with an antibody against GAPDI (lower blot). FIG. 3E. Representative blots are shown. The two isoforms of JNK have observed molecular weights of 46 and 54 kDa. FIG. 3F. Blots from four independent experiments were analyzed by densitometric scanning (Li-Cor Odyssey software) in a range where band intensity was linear. The ratio of the pJNK band to GAPDH was calculated, and the results normalized to control untreated cells. The data are the mean±SD of the four experiments. FIG. 3G. The UV-visible spectrum of Chi(III) in 50 mM potassium phosphate buffer, pH 7.4 was recorded before (black line) and immediately after adding a three-fold molar excess of ascorbate (blue line), cysteine (teal line), or reduced glutathione (GSH, pink line). FIG. 3H. COS-7 cells were incubated for 3 h with 1 mM paraquat, in the absence or presence of 100 μM of the indicated drugs. The cells were counted 48 h later using a hemocytometer. The data are the mean±SD of at least five experiments per condition. Veh, vehicle; Cbi, cobinamide; Imp, imisopasem; MnTP, MnTBAP; CbL, cobalamin; GSH, reduced glutathione. White scale bar in A and Cis 10 μm in length. The data were analyzed by a one-way ANOVA (for Panels B, D, and H, p<0.0001 and for Panel F, p=0.0068) followed by Tukey's multiple comparison test of all conditions; **, ***, and **** indicate p<0.01, 0.001, and 0.0001, respectively for the indicated paired comparisons. In Panels B and D, comparison of vehicle-treated cells to cells treated with cobinamide in the absence or presence of rotenone was not significant, whereas comparison of vehicle-treated cells to rotenone-exposed cells treated with imisopasem, MnTBAP, or cobalamin was significant, with the p values at least <0.001. In Panel F, comparison of control untreated cells to cells receiving H₂O₂ and Cbi(III) or Chi(II) was not significant.

FIGS. 4A-4K. Cobinamide Rescues Flies from Paraquat Poisoning and Prevents Abnormal Lipid and Protein Oxidation, DNA Damage, and Fibrosis in the Hearts of Diabetic Mice. FIG. 4A. At time zero, 10 fruit flies (D. melanogaster) were transferred to vials with food to which either phosphate-buffered saline (PBS), 0.8 mM of the indicated drugs, 20 mM paraquat (PQ), or 20 mM paraquat with 0.8 mM of the indicated drugs had been added. The number of live flies was counted each day for 7 d. The experiment was repeated seven times, for a total of 70 flies per condition. The percent surviving flies was calculated; error bars represent standard deviations and are shown in one direction only for sake of clarity. FIGS. 4B-4K. Twenty-week-old male C57BL/6NHsd mice were injected with saline or streptozotocin, and 13 d later, the mice were randomized to receive plain drinking water or 1 mM histidyl-cobinamide in the drinking water. After three months, they were euthanized and their hearts were flash frozen in liquid nitrogen or fixed in paraformaldehyde. Initially, each group had eight mice, but at the time of euthanasia, three of the streptozotocin-injected mice were found not to be diabetic (one mouse that did not receive cobinamide and two mice that did receive cobinamide); they were excluded from all analyses. The amount of 8-isoprostane (FIG. 4B) was measured by ELISA, protein oxidation (FIGS. 4C-4D) was assessed by immunoblotting, and nitrotyrosine content (FIGS. 4E-4F) was assessed by immunohistochemistry. DNA damage was assessed by 8-OH-deoxyguanosine staining (FIGS. 4G-4H) and a long amplicon qPCR-based assay (FIG. 4I). Cardiac fibrosis (FIGS. 4J-4K) was assessed by Mallory trichrome staining, which stains collagen blue. In Panels F, G, and J, the two adjacent squares are representative areas from two separate mice; the scale bars in Panels E and G are 10 μm and the bar in Panel J is 100 μm. The yellow arrows in Panels E and G indicate positively-stained cells. For Panels F and H, at least 100 cells from each sample were visualized; the data shown are the percent of cells that stained positive. For Panels C, D, and I, frozen heart samples were selected randomly from each of the four groups and were analyzed. For Panel D, blots were analyzed by densitometric scanning using Li-Cor Odyssey software, with the density of each lane normalized to the actin band. For Panel I, DNA was extracted and used as a template for PCR of an 8.7 kb fragment of the β-globin gene. Any DNA injury such as modified bases or strand breaks inhibit DNA synthesis, with the data expressed as the amount of DNA generated over the amount of input DNA. For Panel K, the area of blue staining within the total cross-section of the cardiac apex was measured using Image-Pro Premier software. The data are expressed as the percent blue area within the total heart section and are the mean±SD of hearts from four mice per condition. For Panels F, H, and K, slides from 4-5 mice from each of the four groups were selected randomly and analyzed by an operator blinded to the specific conditions. Chi, cobinamide in Panel A and histidyl-cobinamide in Panels B-K; Imp, imisopasem; MnTP, MnTBAP; CbL, cobalamin; DNPH, 2,4-dinitrophenylhydrazine; ND, non-diabetic; D, diabetic; Veh, vehicle. The data were analyzed by a two-way ANOVA (interaction, p<0.05 for all data shown in bar graphs) followed by Sidak's multiple comparison test; *, **, ***, and **** indicate p<0.05, 0.01, 0.001, and 0.0001, respectively for the indicated paired comparisons. In all cases, comparison of the non-diabetic vehicle versus non-diabetic cobinamide groups and comparison of non-diabetic cobinamide versus diabetic cobinamide groups was not significant.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2nd ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20th ed., (Lippincott, Williams & Wilkins 2003), and Remington, The Science and Practice of Pharmacy, 22th ed., (Pharmaceutical Press and Philadelphia College of Pharmacy at University of the Sciences 2012).

Disclosed herein are compositions and methods for treating oxidative stress, and conditions associated with oxidative stress such as diabetes, in a subject in need thereof with an effective amount of cobinamide. Oxidative stress can be caused by, for example, increased levels of reactive oxygen species (ROS) and reactive nitrogen species (RNS) in the patient.

In embodiments, the disclosure provides a method of treating oxidative stress in a subject with diabetes, where the method includes administering to a subject in need thereof an effective amount of cobinamide, a cobinamide derivative, or a salt thereof. In some embodiments, the diabetes is associated with oxidative stress, which can contribute to the severity of the diabetes, particularly peripheral vascular disease associated with diabetes. In some embodiments, the cobalt atom of the cobinamide, cobinamide derivative, or salt thereof independently may be coordinated with one or more ligands. In some embodiments, the cobinamide, cobinamide derivative, or salt thereof is administered orally.

In embodiments, the disclosure provides a method of treating oxidative stress in a subject, where the method includes administering to a subject in need thereof an effective amount of cobinamide, a cobinamide derivative, or a salt thereof. In some embodiments, the oxidative stress contributes to diabetes, cardiac fibrosis, aortic aneurysm, lipid or protein oxidation, or DNA damage.

In embodiments, the disclosure provides a method of treating oxidative stress associated with diabetes, where the method includes administering to a subject in need an effective amount of a composition comprising an anti-oxidant to lower reactive oxygen species. In some embodiments, the anti-oxidant is cobinamide, a cobinamide derivative, or a salt thereof.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be treated.

As used herein, the term “pharmaceutical composition” refers to pharmaceutically acceptable compositions, where the compositions include one or more cobinamide compounds, and, in some embodiments, also includes a pharmaceutically acceptable carrier. In some embodiments, the pharmaceutical composition is a combination.

As used herein, the term “pharmaceutically acceptable” means approved by a regulator agency of the Federal or a state government or listed in the U.S. Pharmacopoeia, other generally recognized pharmacopoeia in addition to other formulations that are safe for use in animals, and more particularly in humans and/or non-human mammals.

As used herein, the term “pharmaceutically acceptable carrier refers to an excipient, diluent, preservative, solubilizer, emulsifier, adjuvant, and/or vehicle with which one or more cobinamide compounds may be administered. Such carriers may be sterile liquids, such as water and oils, including those of petroleum, animal, vegetable, or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media an agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compounds, such use in the composition is contemplated.

As used herein, the phrases “effective amount,” “therapeutically effective amount,” or the like refer to an amount of one or more cobinamide compounds that is sufficient to prevent, treat or ameliorate, or in some manner reduce the symptoms associated with oxidative stress. For example, an effective amount in reference to oxidative stress is that amount which is sufficient to neutralize, block, or prevent onset of the adverse effects of oxidative stress or if symptoms have begun, to palliate, ameliorate, stabilize, reverse or slow progression of adverse effects, or otherwise reduce pathological consequences of oxidative stress. In any case, an effective amount may be given in single or divided doses.

As used herein, the terms “treatment,” “treating,” or the like embrace at least an amelioration of the symptoms associated with oxidative stress in the patient, where amelioration is used in a broad sense to refer to at least a reduction in the magnitude of a parameter, e.g., a symptom associated with oxidative stress. As such, “treatment,” “treating,” or the like also includes situations where oxidative stress or at least the symptoms associated therewith, are completely inhibited (e.g., prevented from happening) or stopped (e.g., terminated) such that the patient no longer suffers from the adverse effects associated with oxidative stress or at least the symptoms that characterize oxidative stress.

The term “combination” refers to either a fixed combination in one dosage unit form, or a kit of parts for the combined administration where one or more cobinamide compounds and a combination partner (e.g., another drug as explained below, also referred to as a “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof, and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredient, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently, or sequentially with no specific time limits, wherein such administration provides therapeutically effective levels of the two compounds in the body of the patient. The latter also applies to cocktail therapy, e.g., the administration of two or more active ingredients.

As used herein, the phrase “cobinamide compound” refers to cobinamide (e.g., Chi(III)) and/or cobinamide derivatives. A cobalt atom of the cobinamide and cobinamide derivatives independently may be coordinated with two ligands. As used herein, the phrase “cobinamide derivative” refers to a biologically active derivative of cobinamide, such as a heterocylic or heteropolycylic compound that is (i) coordinated to the central cobalt atom, and (ii) substituted with two or more alkyl substituents (e.g., four to eight alkyl substituents) that may include at least one polar functional group, such as an amide, and ester, an ether, carboxylic acid, etc. The heterocyclic or heteropolycyclic compound may include 4 heteroatoms, such as nitrogen, oxygen, etc. As an example, a cobinamide compound may include 5-amino-tetrazole-combinamide, which is cobinamide coordinated with two 5-amino-tetrazole ligands. As used herein, the phrase “amino-tetrazole” refers to a tetrazole moiety substituted at any one or more positions with (i) an amino moiety and/or (ii) a C1-C3 alkyl comprising an amino moiety. As used herein, the phrase “acetyl-tetrazole” refers to a tetrazole moiety substituted at any one or more positions with (i) an acetyl moiety and/or (ii) a C1-C3 alkyl comprising an acetyl moiety.

In some embodiments, the one or more cobinamide derivative include an amino-tetrazole-cobinamide, a di-(amino-tetrazole)-cobinamide, an acetyl-tetrazole-cobinamide, a di-(acetyl-tetrazole)-cobinamide, an acetyl-imidazole-cobinamide, a di-(acetyl-imidazole)-cobinamide, a histidyl-cobinamide, or a combination thereof. An example of an amino-tetrazole-cobinamide is 5-amino-tetrazole-cobinamide. An example of a di-(amino-tetrazole)-cobinamide is di-(5-amino-tetrazole)-cobinamide. An example of an acetyl-tetrazole-cobinamide is 5-acetyl-tetrazole-cobinamide. An example of a di-(acetyl-tetrazole)-cobinamide is di-(5-acetyl-tetrazole)-cobinamide. An example of an acetyl-imidazole-cobinamide is 4-acetyl-imidazole-cobinamide. An example of a di-(acetyl-imidazole)-cobinamide is di-(4-acetyl-imidazole)-cobinamide.

As used herein, “Cbi(III)” refers to cobinamide in the +3 oxidation state, and “Cbi(II)” refers to cobinamide in the +2 oxidation state.

The term “pharmaceutically active” as used herein refers to the beneficial biological activity of a substance on living matter and, in particular, on cells and tissues of the human body. A “pharmaceutically active agent” or “drug” is a substance that is pharmaceutically active and a “pharmaceutically active ingredient” (API) is the pharmaceutically active substance in a drug.

The term “pharmaceutically acceptable salt” as used herein refers to acid addition salts or base addition salts of the compounds, such as the multi-drug conjugates, in the present disclosure. A pharmaceutically acceptable salt is any salt which retains the activity of the parent agent or compound and does not impart any deleterious or undesirable effect on a subject to whom it is administered and in the context in which it is administered. Pharmaceutically acceptable salts may be derived from amino acids including, but not limited to, histidine or cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J Pharm. Sci. 66:1, 1977). In some embodiments, a “pharmaceutically acceptable salt” is intended to mean a salt of a free acid or base of an agent or compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. An agent or compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, [gamma]-hydroxybutyrates, glycolates, tartrates, and mandelates.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

Here it was determined reaction rates of cobinamide in the +3 and +2 oxidation states [cobinamide(III) and cobinamide(II), abbreviated Cbi(III) and Cbi(II), respectively] with O₂·⁻ and H₂O₂ and reaction rates of Chi(II) with ONOO⁻. These rates were compared to those published for cobalamin and the potent superoxide dismutase (SOD) mimetic imisopasem manganese (M40403) and the membrane-permeable SOD mimetic and ONOO⁻ scavenger MnTBAP [manganese(III)tetrakis(4-benzoicacid)porphyrin]²⁴⁻²⁸. Like cobalamin, the latter two compounds have been shown to serve as antioxidants in cells and animals^{26,27,29-32} It was found that the reaction rates of Cbi(III) and Cbi(II) with the oxidizing species were generally comparable to or substantially higher than those for the other three agents, and that cobinamide was considerably better than all three agents at reducing oxidative stress in mammalian cells and fruit flies. Furthermore, cobinamide overcame lipid and protein oxidation, DNA damage, and excessive fibrosis in the hearts of diabetic mice when administered in drinking water at a dose well tolerated by the mice.

It was found that cobinamide serves as a superoxide dismutase and catalase mimetic and that Cbi(II) reacts rapidly with ONOO⁻. These in vitro data translated well to in vivo conditions because it was found cobinamide is a potent antioxidant in mammalian cells and fruit flies, and reduced oxidative stress and DNA damage in a mouse model of diabetic cardiomyopathy.

Many agents have been shown to have antioxidant properties, but some such as α-tocopherol (vitamin E) and resveratrol have low water solubility and others such as N-acetylcysteine do not predominantly neutralize free radicals. To compare cobinamide to similar agents, cobalamin, imisopasem, and MnTBAP were selected for the following reasons. Cobalamin is structurally similar to cobinamide, cobalamin(II) reacts readily with O₂·⁻ and ONOO⁻, both cobalamin(II) and cobalamin(III) react with hydrogen peroxide, and cobalamin has antioxidant effects in several in vivo systems (Table I)^{13,14,17,19-21}. Imisopasem and MnTBAP are SOD mimetics, with imisopasem having a relatively high reaction rate with O₂·⁻, and MnTBAP additionally reacts with ONOO⁻; both agents have been used extensively in cells and animals (Table 1)²⁴⁻³².

TABLE 1

| | | | Hydrogen | | | |
|---|---|---|---|---|---|---|
| | Superoxide | | Peroxide | | Peroxynitrite | Nitric Oxide |
| Compound | (O₂•⁻)(^{a}) | | (H₂O₂)(^{b}) | | (ONOO⁻) | (•NO) |
| Cbi(III) | 1.1 × 10⁸ | (^{c}) | 8.0 × 10² | (^{c}) | Not studied | 2.4 × 10⁸ (51) |
| Cbi(II) | 1.9 × 10⁸ | (^{c}) | 3.7 × 10⁴ | (^{c}) | 6.3 × 10⁶ (^{c}) | Kₐ: 1.3 × 10¹⁰ (51) |
| Cobalamin(III) | 9.4 × 10⁶ | (^{c}) | 4.5 × 10⁶ | (17) | (^{d}) | No activity (51, 71) |
| Cobalamin(II) | 3.8 × 10⁸ | (13) | 1.4 × 10² | (16) | 3.7 × 10⁵ (14) | Kₐ: 1.5 × 10⁸ (51) |
| Imisopasem | 1.6 × 10⁷ | (24) | 8.2 | (25) | No activity (26) | No activity (26) |
| MnTBAP | ~4.0 | (54) | 5.8 | (25) | ~1 × 10⁵ (27) | No activity (28) |

(^{a})Mammalian Cu/Zn SOD 1 and 3: 2-6 × 10⁹ (33, 72); mitochondrial Mn SOD2: 7-10 × 10⁸ (33, 73)
(^{b})Mammalian catalase: 1.5 × 10⁶ (25)
(^{c}) New data presented in this study.
(^{d}) No published data found.

Table I. Reaction Rates of Cobinamide (Cbi), Cobalamin, Imisopasem, and MnTBAP with Reactive Oxygen and Nitrogen Species. Reactions of the indicated compounds with O₂·⁻, ONOO⁻, and ·NO are all second order reactions with rate constants in units of M⁻¹ s⁻¹. The reactions of Cbi(III) and Cbi(II) with H₂O₂ are third order with rate constant units of M⁻² s⁻¹. Chi(II) and cobalamin(II) bind nitric oxide, so a Kₐ is shown in units of M⁻¹. For the new data presented in this study, rate constants were measured for O₂·⁻ using a hypoxanthine-xanthine oxidase-cytochrome c system, for H₂O₂ using a H₂O₂-specific sensor (World Precision Instruments), and for ONOO⁻ using a stopped-flow instrument. Published rate constants for Cu/Zn and Mn superoxide dismutases (SOD) and catalase are shown for reference.^{a,b} Letters and numbers in parenthesis refer to footnotes and references, respectively.

While some variability existed among the three comparator drugs in terms of efficacy—for example, cobalamin was the only one that improved cell growth in paraquat-treated COS-7 cells and MnTBAP was the only one that enhanced fly survival during paraquat exposure—cobinamide was superior to the three agents in all cell and fly experiments. This may be due to cobinamide's generally higher reaction rates with O₂·⁻, H₂O₂, and ONOO⁻ compared to the other agents (Table I). Cobinamide's favorable reaction with O₂·⁻ is likely due to its reduction potential of +270 mV, which is about half-way between the one-electron reduction potential of oxygen (−160 mV) and superoxide (+890 mV), and similar to that of the SOD enzymes (˜+300 mV)⁵⁴. It is also possible that cobinamide enters cells more readily than the other drugs; this could explain why cobinamide effectively neutralized H₂O₂ activation of JNK in H9c2 cells, while cobalamin, which has a reportedly high reaction rate with H₂O₂, had no effect. Either way, cobinamide outperformed the comparator drugs, suggesting it may also be effective in the cell and animal systems of oxidative stress where the other drugs have shown efficacy. Cells and flies were used in these studies because to have done comparator studies in mice would have required an inordinately large number of animals. Flies enabled comparison of the drugs in a whole animal, and moreover, they are easy to handle and are used in drug development⁵⁵.

It was found that Cbi(II) reacted faster with hydrogen peroxide than Cbi(III) (Table I). Consistent with these in vitro data, Cbi(II) was more effective than Cbi(III) at reducing H₂O₂-induced JNK phosphorylation. Cobinamide may exist mainly as Cbi(II) in vivo, because Cbi(III) was reduced quickly to Cbi(II) by ascorbate, cysteine, and GSH. Serum concentrations of ascorbate and cysteine are ˜51 μM and 238 μM, respectively, and the intracellular concentration of GSH is 1-10 mM, depending on the cell type⁵⁶⁻⁵⁸. All of these values are considerably higher than the 0.1-0.5 μM concentration we found in the plasma of mice that had received cobinamide in their drinking water. Moreover, cobalamin is converted intracellularly to its coenzyme forms methyl-cobalamin and adenosyl-cobalamin via enzymatic reduction of cobalamin(III) to cobalamin(II)⁵⁹; Cbi(III) could possibly be reduced by this enzyme system.

Superoxide production by mitochondria occurs mostly in the matrix⁶⁰. Cobinamide's nearly complete elimination of MitoSOX Red-induced fluorescence in rotenone-treated cells suggests cobinamide can enter mitochondria. Similarly, cobinamide's rapid recovery of oxygen consumption in cyanide- and hydrogen sulfide-poisoned cells and animals is also consistent with mitochondrial uptake⁹⁻¹¹. In addition to reducing intramitochondrial O₂·⁻, cobinamide recovered the mitochondrial membrane potential. This was likely because O₂·⁻ activates mitochondrial uncoupling proteins, thereby regulating the membrane potential, and elimination of the O₂·⁻ allows return of the membrane potential³⁹.

Although cobinamide reacts with ·NO (Table I) and ·NO is a major regulator of blood pressure, no change in blood pressure was found in mice treated with cobinamide for several months. Of course, this does not rule out that cobinamide reacted directly with ·NO and thereby could reduce the ·NO concentration, because cobinamide could have simultaneously increased ·NO by neutralizing O₂·⁻; O₂·⁻ reacts with ·NO to generate ONOO⁻ at a diffusion-limited rate that exceeds the reaction rate of superoxide dismutase⁵⁴. ONOO⁻ has been shown to be increased in the hearts of diabetic mice⁴³, and increased ONOO⁻ was a likely cause of the increased tyrosine nitration in the diabetic mice, which was reduced by cobinamide. Although cobinamide can potentially have multiple effects, it reduced nitrosative stress without impairing a physiological function of ·NO.

In a mouse model of human familial aortic aneurysm secondary to a gain-of-function mutation in cGMP-dependent protein kinase, we observed increased oxidative stress in the aortas and aortic media degeneration; administering 1 mM cobinamide in the drinking water, i.e., the same concentration used in the present studies, blocked the increase in oxidative stress and pathological changes, and prevented aneurysm formation⁴⁶. The mice showed no clinical or laboratory evidence of toxicity⁴⁶. When combined with the current data showing that cobinamide reduced markers of oxidative stress and DNA damage in the hearts of diabetic mice, cobinamide has eliminated oxidation-induced changes in tissues of mice in two separate models at a safe, well-tolerated dose. Cobinamide did not act via improved glucose handling in the current study, because the cobinamide-treated diabetic mice showed the same degree of glucose intolerance as non-treated animals.

Several limitations to these studies should be noted. First, a hypoxanthine-xanthine oxidase-cytochrome c system was used as the main method to measure cobinamide's reaction with O₂·⁻. This system can be impacted by concomitantly generated hydrogen peroxide, but catalase was included in the experiments and showed that it eliminated the generated H₂O₂. Moreover, cobinamide's reaction with O₂·⁻ was also measured using the spin trap DMPO, and similar results were found as in the cytochrome c system. Second, when JNK phosphorylation was assessed in H9c2 cells, H₂O₂ was added to the cells to yield a robust and measurable response. It is possible cobinamide reacted with the H₂O₂ prior to entering the cells, but cobinamide would likely also react with intracellular H₂O₂ because it reacted with endogenously-produced O₂·⁻ in cells treated with rotenone. Furthermore, cobalamin(III), with a reportedly high reaction rate with H₂O₂ was without effect, suggesting that neutralization of the H₂O₂ did not occur outside the cell. Third, only male mice were used in the studies of diabetes-induced oxidative stress. Female mice might respond differently to cobinamide, but cobinamide was equally effective in male and female mice with the gain-of-function mutation in cGMP-dependent protein kinase⁴⁶; moreover, in the fly experiments, males and females were used, and they responded similarly to cobinamide's antioxidant effects. Fourth, although compensating for the increased water consumption of the diabetic mice was attempted by decreasing the cobinamide concentration in their drinking water, this was not strictly possible because the mice were housed three to four per cage. Variation in water consumption likely led to the wide variation in plasma cobinamide concentrations among the diabetic mice. Finally, cardiac function was not assessed in the mice, because the study was not powered for echocardiographic measurements. Future studies will include such assessments.

In addition to diabetes, increased oxidative stress occurs in a wide variety of other diseases, for example, cardiovascular disorders such as heart failure and ischemia-reperfusion injury, neurodegenerative diseases such as Alzheimer's disease and multiple sclerosis, and acute and chronic inflammatory conditions such as bacterial or viral infections and rheumatoid arthritis⁶¹⁻⁶³ In several of these diseases, clinical trials of antioxidants have yielded mixed results, possibly due to inadequate drug efficacy^{62,63}. A need exists for more effective antioxidants⁶⁴. Compared to most other antioxidants, cobinamide is unique because it serves as both a superoxide and catalase mimetic, it can scavenge excess nitric oxide, and it reacts with peroxynitrite (Table I). Cobinamide's versatility and potency in neutralizing reactive oxygen and nitrogen species may explain why it was superior to three other antioxidants in the present studies, and suggest it has potential utility in treating diseases characterized by increased oxidative stress.

### Cobinamide Synthesis

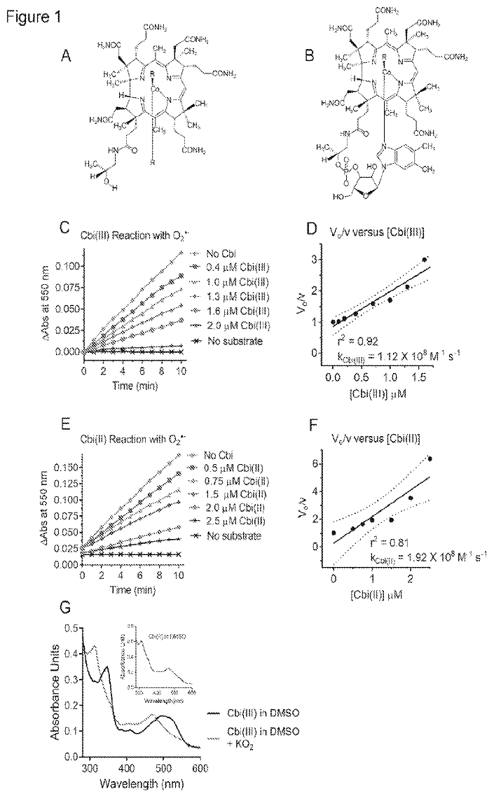

Cobinamide was synthesized from cobalamin by base hydrolysis using freshly made cerium hydroxide (from cerium chloride), and purified over two reversed phase resin columns⁴. The resulting product was >98% pure as determined by high performance liquid chromatography with UV detection and by mass spectrometry. Under ambient conditions, the cobalt is in the +3 oxidation state, and in aqueous solutions at neutral pH, a water and hydroxyl group are coordinated to the cobalt, i.e., it is aquohydroxo-cobinamide (FIG. 1A). Throughout the text, this species is referred to as “cobinamide,” but when it is important to delineate its oxidation state, it is referred to as Cbi(III). To generate Cbi(II), two molar equivalents of either ascorbic acid or sodium borohydride were added to Cbi(III). The ascorbic acid was removed by passing the solution over an anion exchange column (Dowex 1) and the borohydride was decomposed in dilute acid. The resulting Cbi(II) remains in the reduced state for several hours, even on exposure to air. In the mouse experiments, cobinamide was administered as histidyl-cobinamide (cobinamide with two bound histidine molecules) because it is stable in aqueous solutions, allowing it to be used in drinking water. It was generated by adding three molar equivalents of L-histidine to cobinamide.

Measurement of Cobinamide Reaction with Superoxide

The reaction of cobinamide with O₂·⁻ was studied in two different systems, generating O₂·⁻ using a hypoxanthine-xanthine oxidase system.

The first system contained 100 μM hypoxanthine and 0.1 unit xanthine oxidase in 20 mM sodium phosphate buffer, pH 7.1. The amount of O₂·⁻ was measured by following reduction of ferric-cytochrome c (70 μM) at 550 nm as described previously⁶⁵. Catalase was included (32 units per sample), because xanthine oxidase generates both O₂·⁻ and H₂O₂, and the latter can re-oxidize the ferro-cytochrome c product^{34,66}. The reaction was followed for 10 min. A no substrate blank lacking hypoxanthine was included.

The second system contained 1 mM hypoxanthine, 0.04 U xanthine oxidase, and 0.26 M DMPO in phosphate-buffered saline, pH 7.4 (PBS). Samples were transferred to a capillary tube, and introduced into the EPR cavity of a Magnettech MiniScope MS5000. DMPO-OH signals arising from the DMPO-OOH spin adduct were measured at 37° C. for 5 min. The area under the DMPO-OOH peak was calculated using Origin 2022b software. To avoid variability across runs, signal amplitudes were normalized to the intensity of simultaneously recorded Mn reference signals originating from ZnS:Mn²⁺ fixed within the EPR cavity.

In both systems, the experiments were conducted in the absence and presence of the indicated concentrations of Cbi(III) or Chi(II) 65.

### Cobinamide Functions as a Superoxide Dismutase Mimetic

The metal centers of superoxide dismutase are cyclically reduced and oxidized, in the process converting O₂·⁻ to O₂ and H₂O₂³³. Cbi(III) is relatively easily reduced to Chi(II) and the latter can re-oxidize to Cbi(III)^{5,6}. This suggests cobinamide could serve as a superoxide dismutase mimetic according to the following two reactions:

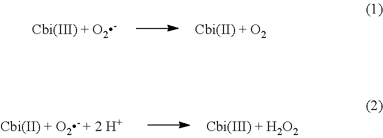

Both Cbi(III) and Cbi(II) reacted readily with O₂·⁻, as measured in a hypoxanthine-xanthine oxidase-cytochrome c reduction system, yielding apparent rate constants of 1.12 and 1.92×10⁸ M⁻¹ s⁻¹ for the Chi(III) and Cbi(II) reactions, respectively (FIG. 1C-F, Table I). Catalase was included in the reaction mixture, because the hypoxanthine-xanthine oxidase system also generates H₂O₂ and the latter can oxidize the ferro-cytochrome c product back to ferric-cytochrome c, thereby interfering with the kinetic measurement³⁴.

The reaction of Chi(III) with O₂·⁻ was also measured using the spin-trap DMPO, which yields a distinctive electron paramagnetic resonance (EPR) spectrum on reacting with O₂·⁻. Sub-micromolar concentrations of cobinamide reduced the EPR signal in a dose-dependent fashion, yielding an apparent rate constant of 7.5×10⁷ M⁻¹ s⁻¹ for the reaction of Chi(III) with O₂·⁻ (FIG. 1B). This value is within experimental error of the value of 1.12×10⁸ M⁻¹ s⁻¹ found in the cytochrome c reduction system.

To study the mechanisms of the reactions and provide evidence for equations (1) and (2), the reactions were followed spectrophotometrically and by visible color change. The reduction of Cbi(III) to Cbi(II) in dimethyl sulfoxide (DMSO) was studied; O₂·⁻ is relatively stable in aprotic solvents and the lack of hydrogen ions prevents re-oxidation of Cbi(II) by O₂·⁻³⁵. Focusing solely on the reaction of Chi(III) with O₂·⁻, it was observed that O₂·⁻ fully reduced Cbi(III) to Cbi(II) (FIG. 1G). To study O₂·⁻ oxidation of Cbi(II) to Chi(III), O₂·⁻ in DMSO was added to an aqueous solution of Cbi(II). The Cbi(II) was rapidly oxidized to Cbi(III) and then largely reduced back to Cbi(II). Thus, it appears cobinamide can serve as a superoxide dismutase mimetic by cycling between Chi(III) and Cbi(II); the reaction rates of Cbi(III) and Cbi(II) with O₂·⁻ are about 10 and many orders of magnitude faster than the reaction rates of imisopasem and MnTBAP with O₂·⁻, respectively (Table I).

Cobalamin (II) has been shown to react with O₂·⁻ at a rate two times faster than for Cbi(II)¹³ (Table I), but the reaction of cobalamin(III) with O₂·⁻ had not been previously reported. Cobalamin (III) does react with O₂·⁻, but at a rate less than one-tenth that of Chi(III) (Table I).

### Cobinamide Functions as a Catalase Mimetic

Like superoxide dismutase, catalase has a metal (iron) center that is alternately reduced and oxidized during the conversion of H₂O₂ to H₂O and O₂ (36). It was hypothesized that cobinamide could function as a catalase mimetic according to reactions (3) and (4):

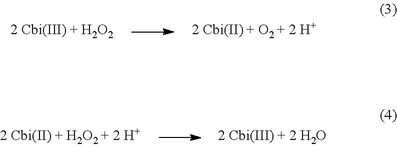

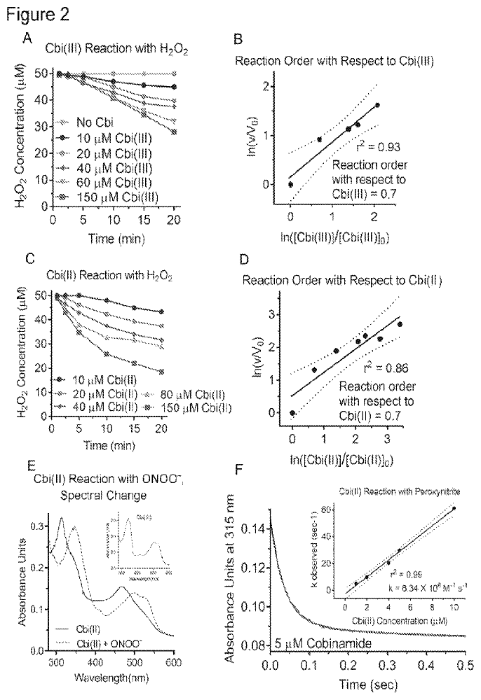

Both Cbi(III) and Chi(II) react with H₂O₂, and the reaction was essentially first order with respect to Cbi(III) and Cbi(II) (FIG. 2A-D), and second order with respect to H₂O₂ for both cobinamide species. Using these data, the apparent rate constants were calculated to be 8.00×10² and 3.68×10₄ M⁻² s⁻¹ for Cbi(III) and Chi(II), respectively (Table I). Because the overall rate constants are third order, it is not possible to compare them strictly with published rate constants for the reaction of H₂O₂ with imisopasem, MnTBAP, and cobalamin shown in Table I. To assess if cobinamide was acting via equations (3) and (4), similar experiments were performed to those done with O₂⁻. The reactions of H₂O₂ with Chi(III) and Chi(II) generated in a concentration and time-dependent fashion largely Cbi(II) and Cbi(III), respectively, although the final products were intermediate species. Thus, cobinamide appears to function as a catalase mimetic.
Cbi(II) Reacts with Peroxynitrite

Peroxynitrite (ONOO⁻) is a potent oxidizing agent that has been shown to oxidize cobalamin(II) to cobalamin(III) 14.37. ONOO⁻ oxidizes Cbi(II) rapidly to Chi(III) (FIG. 2E). In assessing the rate of the reaction in stopped-flow experiments, and an apparent rate constant of 6.34×10⁶ M⁻¹ s⁻¹, or more than 10- and 50-fold higher than the reactions of cobalamin(II) and MnTBAP with ONOO⁻, respectively, was observed (FIG. 2F, Table I).

### Assessment of Mitochondrial Superoxide Content

H9c2 cells were plated on glass cover slips in 24 well dishes, and 16 h later the medium was changed to phenol red-free DMEM supplemented with 20 mM Hepes, 0.1% fetal bovine serum, and 0.5% bovine serum albumin. The cells were incubated for 30 min at room temperature with 5 μM rotenone in the absence or presence of 2.5 μM of the indicated anti-oxidant, with 5 μM MitoSOX Red added during the last 10 min. MitoSOX Red is a dihydroethidium derivative containing triphenyl-phosphonium, which localizes it to respiring mitochondria. Its reaction with O₂·⁻ yields 2-hydroxy-mitoethidium (2-OH-Mito-E″), whereas its reaction with other oxidative species yields mitoethidium (Mito-E⁺)⁶⁷. These two oxidative derivatives have overlapping fluorescence spectra, but they can be distinguished by high performance liquid chromatography (HPLC)⁶⁸. Rotenone increased 2-OH-Mito-E⁺ about two-fold, without increasing Mito-E⁺. Thus, any increased fluorescence observed on treating cells with rotenone could be ascribed to an increase in 2-OH-Mito-E⁺, and decreased fluorescence by an anti-oxidant in rotenone-treated cells was likely from a decrease in 2-OH-Mito-E⁺.

### Assessment of Mitochondrial Membrane Potential

For assessment of the mitochondrial membrane potential, H9c2 cells were pre-incubated with 10 μM JC-1 for 10 min at room temperature; JC-1 is a cationic dye that exhibits potential-dependent accumulation in mitochondria indicated by a fluorescence emission shift from green to red⁴⁰. The cells were then washed once with PBS, placed back in the DMEM experimental medium, and incubated with the indicated drugs for 30 min. At the end of the incubation period, nuclei of both the MitoSOX Red- and JC-1-treated cells were stained with 2 mg/ml 4′,6-diamidino-2-phenyl-indole (DAPI) for 3 min, washed once with PBS, and returned to the DMEM experimental medium. The cover slips were removed from the wells, mounted in the DMEM experimental medium, and red (MitoSOX Red and JC-1 aggregates), green (JC-1 monomers), and blue (DAPI) fluorescence intensity was assessed using a Keyence BZ-X700 fluorescence microscope at the following paired excitation/emission wavelengths, respectively: 560/630, 490/525, and 360/460 nm. It was shown in control experiments that cobinamide did not interfere with the fluorescence signal by adding cobinamide to the cells immediately before observing fluorescence.

Cobinamide Rescues Cells from Oxidative Stress, and is Superior to Cobalamin, Imisopasem, and MnTBAP

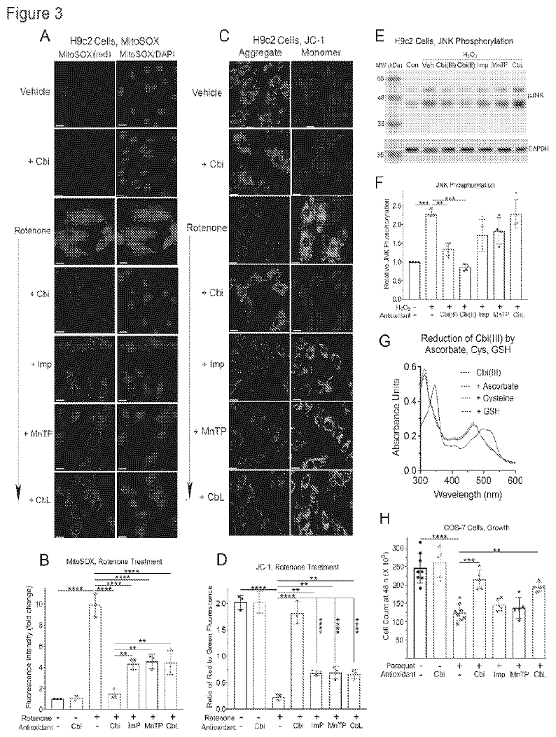

To assess if cobinamide reduces oxidative stress in cells and to compare its in vivo efficacy to that of cobalamin, imisopasem, and MnTBAP, several sets of experiments were performed. First, whether cobinamide reduces mitochondrial-generated O₂·⁻ was evaluated using the fluorescent probe MitoSOX Red. H9c2 rat embryonal cardiomyocytes were treated with rotenone, an inhibitor of complex I, to increase mitochondrial O₂·⁻. Minimal fluorescence was observed in vehicle-treated cells with no change on adding cobinamide, but a marked increase in fluorescence occurred in rotenone-treated cells (FIGS. 3A-3B). Rotenone increased 2-hydroxy-mitoethidium without increasing mitoethidium, indicating that the drug mainly increased O₂·⁻ and not other species that oxidized MitoSOX. Cobinamide markedly reduced fluorescence in rotenone-treated cells to a signal indistinguishable from that found in vehicle-treated cells (FIGS. 3A-3B). Imisopasem, MnTBAP, and cobalamin also reduced the fluorescent signal substantially in rotenone-treated cells compared to vehicle-treated cells, but the signal remained significantly higher than in cells receiving cobinamide (FIGS. 3A-3B; drugs were present at 2.5 μM). Cobinamide's beneficial effect was not due to directly binding, and thereby scavenging, rotenone because no change in the UV-visible spectrum of cobinamide was observed in the presence of an eight-fold excess of rotenone. Monitoring the UV-visible spectrum of cobinamide is a sensitive means to assess ligand binding³⁸. Nor did cobinamide appear to affect mitochondrial mass, because alone it did not significantly change the amount of 2-hydroxy-mitoethidum or mitoethidium.

Increased mitochondrial O₂·⁻ by rotenone would be expected to reduce the mitochondrial membrane potential (ΔΨm), due in part to activation of uncoupling proteins³⁹. The dye JC-1 was used to assess ΔΨm: JC-1 aggregates and shows red fluorescence in mitochondria with high ΔΨm and stays as a monomer with green fluorescence in mitochondria with low ΔΨm⁴⁰. A high red to green fluorescence ratio was observed in vehicle-treated H9c2 cells with no change on adding cobinamide, indicating a relatively high ΔΨm in the cells (FIGS. 3C-3D). Rotenone caused an almost complete loss of red fluorescence and a striking increase in green fluorescence in the cells, indicating a marked decrease in ΔΨm (FIGS. 3C-3D). Cobinamide restored the ratio of red to green fluorescence in rotenone-treated cells to a value indistinguishable from that in vehicle-treated cells, indicating a return of ΔΨm (FIGS. 3C-3D). Imisopasem, MnTBAP, and cobalamin all showed modest degrees of recovery of red to green fluorescence in rotenone--treated cells; as with the measurement of O₂·, the three drugs did not return fluorescence to values found in vehicle-treated cells and cobinamide was significantly better than the three drugs (FIGS. 3C-3D; the four drugs were present at 2.5 μM).

Next, the effect of cobinamide and the comparator drugs was tested on hydrogen peroxide-induced oxidative stress. H9c2 cells were treated with H₂O₂ and phosphorylation/activation of Jun N-terminal kinase was assessed [JNK, also known as stress-activated protein kinase (SAPK)]; JNK is activated downstream of a variety of reactive oxygen species, including H₂O₂¹⁰. Treatment with H₂O₂ for 30 min increased JNK phosphorylation 2.4-fold, and both Cbi(III) and Cbi(II) reduced JNK phosphorylation to a level that was indistinguishable from vehicle-treated cells (FIGS. 3E-3F). Higher cobinamide concentrations (100 μM) were used in these studies than in the rotenone studies, because cobinamide reacts less readily with H₂O₂ than with O₂·⁻ (Table I). Imisopasem and MnTBAP had small non-significant effects and cobalamin had no effect on JNK phosphorylation (FIGS. 3E-3F; the drugs were at 100 μM). The cobalamin was in the +3 oxidation state, i.e., cobalamin(III); cobalamin(II) could not be tested because in the presence of oxygen, it rapidly oxidized back to cobalamin(III). In the absence of H₂O₂, neither Cbi(III), Cbi(II) nor the other three drugs affected JNK phosphorylation or total cellular JNK. It was observed that Cbi(III) is readily reduced to Cbi(II) by ascorbate, cysteine, and reduced glutathione (GSH) under physiological conditions (FIG. 3G) [previous workers have also found that GSH reduces Cbi(III) to Cbi(II)⁴¹].

Finally, whether cobinamide could alleviate the effects of oxidative stress induced by yet another mechanism in a different cell type was tested using paraquat-treated COS-7 cells. Paraquat generates O₂·⁻ through a redox cycling mechanism and increases both intracellular O₂·⁻ and H₂O₂⁴². Exposing COS-7 cells for 3 h to 1 mM paraquat reduced cell growth by ˜50%, when measured 48 h later (FIG. 3H). Cobinamide alone had no effect on cell growth and when added to paraquat-treated cells, it restored growth to ˜80% of the control value (FIG. 3H). Cobalamin also improved cell growth, but it was not as effective as cobinamide, and neither imisopasem nor MnTBAP had an effect (FIG. 3H). As in the studies with rotenone, cobinamide did not appear to be acting by directly binding and scavenging paraquat, because no change was found in the UV-visible spectrum of cobinamide at paraquat concentrations eight times higher than that of cobinamide.

Cobinamide Rescues Flies from Paraquat Poisoning

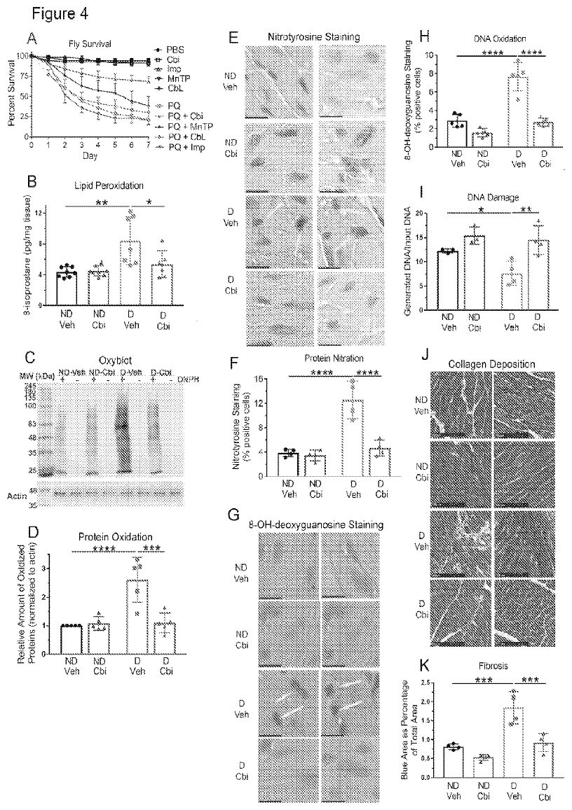

To test cobinamide in a whole animal, D. melanogaster was selected for initial studies, and oxidative stressed was increased in the files by administering paraquat in their food. Cobinamide was again compared to imisopasem, MnTBAP, and hydroxocobalamin, all administered in food. Administration of 0.8 mM of the drugs had no effect on the flies (FIG. 4A). Previous works have found that D. melanogaster tolerate relatively high paraquat concentrations⁴³, and here it was observed that 20 mM paraquat was required to yield >75% mortality over 7 d (FIG. 4A; p<0.0001 by both Mantel-Cox log rank test and area under the curve analysis for comparison between untreated and paraquat-treated flies). Providing 0.8 mM cobinamide with the paraquat reduced mortality to <30%, while 0.8 mM MnTBAP had a lesser effect and 0.8 mM imisopasem and cobalamin had no significant effect (FIG. 4A; for cobinamide, p<0.0001 by both log rank test and area under the curve analysis, and for MnTBAP, p<0.01 by log rank test and <0.0001 by area under the curve analysis for comparison to flies exposed to paraquat in the absence of drugs).

### Generation and Treatment of Diabetic Mice

Male C57BL/6NHsd mice (20 weeks old) were housed three to four animals per cage in a temperature-controlled environment with a 12-h light/dark cycle and fed standard rodent chow with ad libitum access to food and water. After one week of acclimatization, mice weighing 30±3 g were injected intraperitoneally for five consecutive days with either saline (n=20) or 50 mg/kg streptozotocin (n=20) 69. Thirteen days after the last streptozotocin injection, the blood glucose concentration was measured after a 6 h fast using a commercial glucometer; 16 of the 20 streptozotocin-injected mice had a blood glucose concentration >270 mg/mL and were considered diabetic. The 16 diabetic mice and a corresponding number of the saline-injected, non-diabetic mice were split randomly in half to receive either plain drinking water or histidyl-cobinamide in the drinking water. The four groups of animals are referred to as: diabetic vehicle (D-Veh); diabetic, cobinamide-treated (D-Cbi); non-diabetic, vehicle (ND-Veh); and non-diabetic, cobinamide-treated (ND-Cbi). The histidyl-cobinamide concentration was 1 mM for the ND-Cbi mice, but because the diabetic mice drank more than the non-diabetic mice, the cobinamide concentration for the D-Cbi mice was decreased accordingly. The water was replenished twice a week.

After three months of treatment, the mice were weighed, fasted for 6 h, and then underwent an intraperitoneal glucose tolerance test (IPGTT) using 2 g/kg of a 20% glucose solution. Blood glucose concentrations were measured just before the glucose injection (time 0, fasting specimen) and at 15, 30, 60, and 120 min following the injection. One mouse in the D-Veh group and two mice in the D-Cbi group were found not to be diabetic, with a normal fasting blood glucose concentration and normal IPGTT; they were excluded from further study. Two days later, the mice were euthanized by inducing deep anesthesia with 200 mg/kg ketamine and 40 mg/kg xylazine administered by intraperitoneal injection, followed by exsanguination via an open cardiac puncture. The hearts were removed quickly and dipped into ice-cold PBS to remove excess blood; the apex was cut off and fixed in 4% formaldehyde, with the remainder of the heart flash frozen in liquid nitrogen. Paraffin-embedded blocks were made from the formaldehyde-fixed apical samples and cut into 5 μM thick sections that were mounted on glass slides.

Assessment of Lipid 8-isoprostane, Protein Nitrosylation and Carbonylation, DNA Damage, and Collagen Deposition in Heart Samples

8-isoprostane in heart extracts was measured by ELISA. Frozen heart pieces (10-20 mg) were pulverized in liquid nitrogen, and then processed according to the manufacturer's recommendation. Standard curves were included in each assay over a range bracketing sample values. Each sample was measured in duplicate at two different dilutions. The data are expressed as picogram per milligram of wet tissue.

8-OH-deoxyguanosine and nitrotyrosine were assessed by immunohistochemistry. Slides with mounted cardiac sections were incubated for 10 min in 10 mM sodium citrate buffer, pH 6.0 at 80-85° C. Endogenous peroxidase activity was quenched in 3% H₂O₂ for 10 min. After blocking with 2% normal goat serum, slides were incubated overnight at 4° C. with an anti-8-OH-deoxyguanosine or anti-nitrotyrosine primary antibody, followed by a horseradish peroxidase-conjugated secondary antibody. After development with 3-diaminobenzidine (Vector Laboratories)⁴⁶, nuclei were counterstained with hematoxylin, and images were analyzed with a Hamamatsu Nanozoomer Slide scanning system. The number of brown-stained cells were counted from five separate areas of 100 cells per area.

Protein carbonylation was assessed using the Oxyblot protein oxidation system from EMD Millipore. Frozen tissue was pulverized in liquid nitrogen and extracted in RIPA buffer containing 50 mM dithiothreitol and protease inhibitors, with half of the extract (˜5 μg protein) incubated with 2,4-dinitrophenylhydrazine (DNP). The samples were subjected to PAGE-immunoblotting, with carbonylated proteins detected using an anti-DNP antibody.

DNA damage was measured in an 8.7 kb fragment of the mouse β-globin gene using a long-amplicon, quantitative polymerase chain reaction-based assay^{48,49}. Briefly, 10-20 mg of frozen heart tissue was pulverized on dry ice, and large genomic DNA was purified using Qiagen Genomic-tip 20/G columns as recommended by the manufacturer. The DNA was quantified using a nanodrop spectrophotometer, diluted to 6 μg/μL and then quantified again using Quant-iT PicoGreen dsDNA Assay Kit (Invitrogen). The DNA was diluted to 1 ng/μL, and 5 and 10 ng were added to a polymerase chain reaction containing 20 μmol each of 5′ and 3′ primers, and LongAmp Hot Start Taq Master Mix (New England Biolabs, Inc.). Cycling conditions consisted of 94° C. for 2 min, followed by 27 cycles of 94° C. for 15 sec and 63° C. for 12 min. The PCR product was subjected to electrophoresis on a 0.5% agarose gel containing ethidium bromide and was quantified using a LiCor imaging system. Any DNA damage including modified bases and strand breaks halt synthesis such that the amount of generated DNA is inversely proportional to the amount of damage.

Collagen deposition was assessed by staining slides containing mounted cardiac sections with Mallory's trichrome stain, which stains collagen fibers blue. The percent blue area in a total heart section was measured using Image-Pro Premier software (Version 9.0, Media Cybernetics).

Cobinamide Prevents Abnormal Lipid and Protein Oxidation, DNA Damage, and Fibrosis in the Hearts of Diabetic Mice

In the final set of experiments, whether cobinamide could reduce oxidative stress in a mammal exhibiting a common human disease was determined. Diabetes leads to increased oxidative stress in many organs, including the heart where the increased oxidative stress causally contributes to a well-described cardiomyopathy^{44,45}. Type I diabetic mice were generated using streptozotocin, and then provided cobinamide in the drinking water for three months. At the time of euthanasia, the plasma cobinamide concentration in mice that had received cobinamide in their drinking water was 0.11±0.07 μM for non-diabetic mice and 0.55±0.36 μM for diabetic mice. The difference between the two groups was not significant, but the higher values in the diabetic mice were likely due to their increased water consumption. Cobinamide was not measurable in the plasma of mice that had not received the drug.

Over the three-month treatment period, the non-diabetic mice gained an average of 4.1±0.8 g. while the diabetic mice lost an average of 0.3 g, with wide variation in weight change among the latter mice. Cobinamide was well tolerated by both the non-diabetic and diabetic mice, and did not significantly affect weight in either group, although the cobinamide-treated diabetic mice gained an average of 0.63 g, with wide variation among individual mice. Two days prior to euthanasia, a fasting blood glucose and glucose tolerance test showed that the cobinamide-treated mice exhibited the same degree of glucose intolerance as mice that did not receive cobinamide. Cobinamide had no effect on liver function and renal function tests in the mice; complete blood counts could not be performed due to lack of sufficient blood volume, but cobinamide did not affect hematological parameters in mice that received the same amount of cobinamide for six months⁴⁶.

In the hearts of the diabetic mice, increased lipid and protein oxidation were found, as evidenced by increased 8-isoprostane content and protein carbonylation and nitrotyrosine staining, respectively: although some variability existed among the individual diabetic mice, overall they showed a 2.1-, 2.6- and 3.2-fold increase over the non-diabetic mice for 8-isoprostane (FIG. 4B), protein carbonylation (FIGS. 4C-4D), and nitrotyrosine, respectively (FIG. 4E-4F). Previous workers have found increased 8-isoprostane and ONOO⁻ in hearts of diabetic mice^{44,47}; the ONOO⁻-derived nitrogen dioxide radical can cause nitration of protein tyrosine residues³⁷. Evidence was also found for DNA damage in the hearts of the diabetic mice, as evidenced by increased 8-OH-deoxyguanosine staining (FIGS. 4G-4H) and decreased DNA integrity using a long-amplicon polymerase chain reaction-based assay (FIG. 4I)^{48,49}. Although the latter assay is not specific for DNA oxidation, the method is quantitative and highly sensitive, and is well-suited for tissue samples. Cobinamide treatment of the diabetic mice significantly reduced all markers of oxidative stress and DNA damage to values similar to those found in the non-diabetic mice (FIGS. 4B-4I). In all of the above assays, no correlation between the plasma cobinamide concentration and the degree of efficacy was observed in individual mice, but this could have been due to relatively small numbers.

Oxidative stress in diabetes contributes to cardiac fibrosis^{44,45}, and abnormal collagen deposition was found in the hearts of the diabetic mice (FIGS. 4J-4K). As observed by others, some of the collagen was present in perivascular regions (FIG. 4J)⁵⁰. Cobinamide reduced collagen content in the hearts of the diabetic mice essentially to that of non-diabetic mice (FIGS. 4J-4K).

Cobinamide does not Increase Mouse Blood Pressure at the Doses Used

It has previously been shown that nitric oxide (NO) reduces Chi(III) to Chi(II), and that Cbi(II) binds ·NO with high affinity⁵¹ (Table I). Hence, each cobinamide molecule can neutralize two ·NO molecules, and cobinamide can scavenge excess ·NO, both in vitro and in vivo^{52,53}. It seemed possible, therefore, that the cobinamide administered to the mice could have increased their blood pressure, since ·NO is a potent vasodilator. Because diabetes can increase blood pressure, these studies were performed on a parallel set of non-diabetic mice of the same strain and sex and using the same concentration of histidyl-cobinamide in the drinking water as in the above-described studies. No difference in the blood pressure of the mice that received cobinamide was observed as compared to those that did not receive cobinamide.

The work described herein can be found at least in part in Chang et al., Cobinamide is a strong and versatile antioxidant that overcomes oxidative stress in cells, flies, and diabetic mice, PNAS Nexus, 14 Sep. 2022, 1, 1-13.

### REFERENCES

1. Scott, A. I., and Roessner, C. A. (2002) Biosynthesis of cobalamin (vitamin B(12)). Biochem Soc Trans 30, 613-620
2. Hayward, G. C., Hill, H. A. O., Pratt, J. M., Vanston, N. J., and Williams, A. R. W. (1965) The chemistry of vitamin B(12). Part IV. The thermodynamic trans-effect. J. Chem. Soc, 6485-6493
3. Brenner, M., Kim, J. G., Mahon, S. B., Lee, J., Kreuter, K. A., Blackledge, W., Mukai, D., Patterson, S., Mohammad, O., Sharma, V. S., and Boss, G. R. (2009) Intramuscular cobinamide sulfite in a rabbit model of sublethal cyanide toxicity. Ann. Emerg. Med 55, 352-362
4. Chan, A., Jiang, J., Fridman, A., Guo, L. T., Shelton, G. D., Liu, M. T., Green, C., Haushalter, K. J., Patel, H. H., Lee, J., Yoon, D., Burney, T., Mukai, D., Mahon, S. B., Brenner, M., Pilz, R. B., and Boss, G. R. (2015) Nitrocobinamide, a new cyanide antidote that can be administered by intramuscular injection. J. Med. Chem 58, 1750-1759
5. Lexa, D., Saveant, J. M., and Zickler, J. (1980) Electrochemistry of vitamin B12. 6. Diaquocobinamide J Am Chem Soc 102, 4851-4852
6. Lexa, D., Saveant, J. M., and Zickler, J. (1977) Electrochemistry of vitamin B12. 2. Redox and acid-base equilibria in the B12a/B12r system. J Am Chem Soc 99, 2786-2790
7. Chan, A., Balasubramanian, M., Blackledge, W., Mohammad, O. M., Alvarez, L., Boss, G. R., and Bigby, T. D. (2010) Cobinamide is superior to other treatments in a mouse model of cyanide poisoning. Clin. Toxicol. (Phila) 48, 709-717
8. Bebarta, V., Tanen, D., Boudrea, S., Castaneda, M., Zarzabal, L., Vargas, T., and Boss, G. (2014) Intravenous cobinamide versus hydroxocobalamin for acute treatment of severe cyanide poisonong in a swine (Sus Scrofa) model. Ann. Emerg. Med 64, 612-619
9. Brenner, M., Mahon, S. B., Lee, J., Kim, J., Mukai, D., Goodman, S., Kreuter, K. A., Ahdout, R., Mohammad, O., Sharma, V. S., Blackledge, W., and Boss, G. R. (2010) Comparison of cobinamide to hydroxocobalamin in reversing cyanide physiologic effects in rabbits using diffuse optical spectroscopy monitoring. J. Biomed. Opt 15, 017001
10. Jiang, J., Chan, A., Ali, S., Saha, A., Haushalter, K. J., Lam, W. L., Glasheen, M., Parker, J., Brenner, M., Mahon, S. B., Patel, H. H., Ambasudhan, R., Lipton, S. A., Pilz, R. B., and Boss, G. R. (2016) Hydrogen sulfide-mechanisms of toxicity and development of an antidote. Sci. Rep 6, 20831
11. Broderick, K. E., Potluri, P., Zhuang, S., Scheffler, I. E., Sharma, V. S., Pilz, R. B., and Boss, G. R. (2006) Cyanide detoxification by the cobalamin precursor cobinamide. Exp. Biol. Med 231, 641-651
12. Gunasekar, P. G., Sun, P. W., Kanthasamy, A. G., Borowitz, J. L., and Isom, G. E. (1996) Cyanide-induced neurotoxicity involves nitric oxide and reactive oxygen species generation after N-methyl-D-aspartate receptor activation. J. Pharmacol. Exp. Ther 277, 150-155
13. Dassanayake, R. S., Cabelli, D. E., and Brasch, N. E. (2013) Pulse radiolysis studies on the reaction of the reduced vitamin B12 complex cob(II)alamin with superoxide. Chembiochem 14, 1081-1083
14. Mukherjee, R., and Brasch, N. E. (2011) Mechanistic studies on the reaction between cob(II)alamin and peroxynitrite: evidence for a dual role for cob(II)alamin as a scavenger of peroxynitrous acid and nitrogen dioxide. Chemistry 17, 11805-11812
15. Dassanayake, R. S., Farhath, M. M., Shelley, J. T., Basu, S., and Brasch, N. E. (2016) Kinetic studies on the reaction of cob(II)alamin with hypochlorous acid: Evidence for one electron oxidation of the metal center and corrin ring destruction. J Inorg Biochem 163, 81-87
16. Salnikov, D. S., Makarov, S. V., and Koifman, O. I. (2021) The radical versus ionic mechanisms of reduced cobalamin inactivation by tert-butyl hydroperoxide and hydrogen peroxide in aqueous solition. New J. Chem. 45, 535-543
17. Lehene, M., Plesa, D., Ionescu-Zinca, S., Iancu, S. D., Leopold, N., Makarov, S. V., Branzanic, A. M. V., and Silaghi-Dumitrescu, R. (2021) Adduct of aquacobalamin with hydrogen peroxide. Inorg Chem 60, 12681-12684
18. Dassanayake, R. S., Shelley, J. T., Cabelli, D. E., and Brasch, N. E. (2015) Pulse radiolysis and ultra-high-performance liquid chromatography/high-resolution mass spectrometry studies on the reactions of the carbonate radical with vitamin B12 derivatives. Chemistry 21, 6409-6419
19. Moreira, E. S., Brasch, N. E., and Yun, J. (2011) Vitamin B12 protects against superoxide-induced cell injury in human aortic endothelial cells. Free Radic Biol Med 51, 876-883
20. Chan, W., Almasieh, M., Catrinescu, M. M., and Levin, L. A. (2018) Cobalamin-associated superoxide scavenging in neuronal cells Is a potential mechanism for vitamin B12-deprivation optic neuropathy. Am J Pathol 188, 160-172
21. Li, F., Bahnson, E. M., Wilder, J., Siletzky, R., Hagaman, J., Nickekeit, V., Hiller, S., Ayesha, A., Feng, L., Levine, J. S., Takahashi, N., and Maeda-Smithies, N. (2020) Oral high dose vitamin B12 decreases renal superoxide and post-ischemia/reperfusion injury in mice. Redox Biol 32, 101504
22. Birch, C. S., Brasch, N. E., McCaddon, A., and Williams, J. H. (2009) A novel role for vitamin B(12): Cobalamins are intracellular antioxidants in vitro. Free Radic. Biol. Med 47, 184-188
23. van de Lagemaat, E. E., de Groot, L., and van den Heuvel, E. (2019) Vitamin B12 in relation to oxidative stress: A systematic review. Nutrients 11
24. Muscoli, C., Cuzzocrea, S., Riley, D. P., Zweier, J. L., Thiemermann, C., Wang, Z. Q., and Salvemini, D. (2003) On the selectivity of superoxide dismutase mimetics and its importance in pharmacological studies. Br J Pharmacol 140, 445-460
25. Tovmasyan, A., Maia, C. G., Weitner, T., Carballal, S., Sampaio, R. S., Lieb, D., Ghazaryan, R., Ivanovic-Burmazovic, I., Ferrer-Sueta, G., Radi, R., Reboucas, J. S., Spasojevic, I., Benov, L., and Batinic-Haberle, I. (2015) A comprehensive evaluation of catalase-like activity of different classes of redox-active therapeutics. Free Radic Biol Med 86, 308-321
26. Salvemini, D., Mazzon, E., Dugo, L., Riley, D. P., Serraino, I., Caputi, A. P., and Cuzzocrea, S. (2001) Pharmacological manipulation of the inflammatory cascade by the superoxide dismutase mimetic, M40403. Br J Pharmacol 132, 815-827
27. Batinic-Haberle, I., Cuzzocrea, S., Reboucas, J. S., Ferrer-Sueta, G., Mazzon, E., Di Paola, R., Radi, R., Spasojevic, I., Benov, L., and Salvemini, D. (2009) Pure MnTBAP selectively scavenges peroxynitrite over superoxide: comparison of pure and commercial MnTBAP samples to MnTE-2-PyP in two models of oxidative stress injury, a SOD-specific Escherichia coli model and carrageenan-induced pleurisy. Free Radic Biol Med 46, 192-201
28. Szabo, C., Day, B. J., and Salzman, A. L. (1996) Evaluation of the relative contribution of nitric oxide and peroxynitrite to the suppression of mitochondrial respiration in immunostimulated macrophages using a manganese mesoporphyrin superoxide dismutase mimetic and peroxynitrite scavenger. FEBS Lett 381, 82-86
29. Day, B. J., Shawen, S., Liochev, S. I., and Crapo, J. D. (1995) A metalloporphyrin superoxide dismutase mimetic protects against paraquat-induced endothelial cell injury, in vitro. J Pharmacol Exp Ther 275, 1227-1232
30. Masini, E., Cuzzocrea, S., Mazzon, E., Marzocca, C., Mannaioni, P. F., and Salvemini, D. (2002) Protective effects of M40403, a selective superoxide dismutase mimetic, in myocardial ischaemia and reperfusion injury in vivo. Br J Pharmacol 136, 905-917
31. Yim, S. K., Kim, K. M., Lee, C. H., Song, E. K., Lee, S. O., Kim, S. W., Kim, I. H., Kim, S. H., Seo, S. Y., and Lee, S. T. (2021) The superoxide dismutase mimetic M40403, improves 5-fluorouracil-induced small intestinal mucositis in a mouse model. In Vivo 35, 1485-1497
32. Thompson, J. S., Chu, Y., Glass, J., Tapp, A. A., and Brown, S. A. (2010) The manganese superoxide dismutase mimetic, M40403, protects adult mice from lethal total body irradiation. Free Radic Res 44, 529-540
33. Gray, B., and Carmichael, A. J. (1992) Kinetics of superoxide scavenging by dismutase enzymes and manganese mimics determined by electron spin resonance. Biochem J 281 (Pt 3), 795-802
34. Vandewalle, P. L., and Petersen, N. O. (1987) Oxidation of reduced cytochrome c by hydrogen peroxide. Implications for superoxide assays. FEBS Lett 210, 195-198
35. Hayyan, M., Hashim, M. A., and AlNashef, I. M. (2016) Superoxide ion: generation and chemical implications. Chem Rev 116, 3029-3085
36. Goyal, M. M., and Basak, A. (2010) Human catalase: looking for complete identity. Protein Cell 1, 888-897
37. Szabo, C., Ischiropoulos, H., and Radi, R. (2007) Peroxynitrite: biochemistry, pathophysiology and development of therapeutics. Nat. Rev. Drug Discov 6, 662-680
38. Pratt, J. M. (1972) Inorganic chemistry of vitamin B12, Academic Press, London—New York
39. Echtay, K. S., Roussel, D., St-Pierre, J., Jekabsons, M. B., Cadenas, S., Stuart, J. A., Harper, J. A., Roebuck, S. J., Morrison, A., Pickering, S., Clapham, J. C., and Brand, M. D. (2002) Superoxide activates mitochondrial uncoupling proteins. Nature 415, 96-99
40. Smiley, S. T., Reers, M., Mottola-Hartshorn, C., Lin, M., Chen, A., Smith, T. W., Steele, G. D., Jr., and Chen, L. B. (1991) Intracellular heterogeneity in mitochondrial membrane potentials revealed by a J-aggregate-forming lipophilic cation JC-1. Proc Natl Acad Sci USA 88, 3671-3675
41. Dereven'kov, I. A., Makarov, S. V., Thi, T. T. B., Makarova, A. S., Koifman, O. I. (2018) Studies on the reduction of dehydroascorbic acid by glutathione in the presence of aquahydroxocobinamide. Eur. J. Inorg. Chem., 2987-2992
42. Cocheme, H. M., and Murphy, M. P. (2008) Complex I is the major site of mitochondrial superoxide production by paraquat. J Biol Chem 283, 1786-1798
43. Murthy G, P. L., BC; Ravishankar H G; Raj C G, D; Kumar, R. (2018) Evaluation of neuroprotection and antioxidant activities via drosophila model system in the active principle derived from Sida Glutinosa Comm. Ex Cav.—an aboriginal ethno-medicinal plant drug practiced in the folklore medicinal system. BJSTR 11, 8855
44. Hamblin, M., Friedman, D. B., Hill, S., Caprioli, R. M., Smith, H. M., and Hill, M. F. (2007) Alterations in the diabetic myocardial proteome coupled with increased myocardial oxidative stress underlies diabetic cardiomyopathy. J Mol Cell Cardiol 42, 884-895
45. Jia, G., Hill, M. A., and Sowers, J. R. (2018) Diabetic cardiomyopathy: an update of mechanisms contributing to this clinical entity. Circ Res 122, 624-638
46. Schwaerzer, G. K., Kalyanaraman, H., Casteel, D. E., Dalton, N. D., Gu, Y., Lee, S., Zhuang, S., Wahwah, N., Schilling, J. M., Patel, H. H., Zhang, Q., Makino, A., Milewicz, D. M., Peterson, K. L., Boss, G. R., and Pilz, R. B. (2019) Aortic pathology from protein kinase G activation is prevented by an antioxidant vitamin B12 analog. Nat Commun 10, 3533
47. Mariappan, N., Elks, C. M., Sriramula, S., Guggilam, A., Liu, Z., Borkhsenious, O., and Francis, J. (2010) NF-kappaB-induced oxidative stress contributes to mitochondrial and cardiac dysfunction in type II diabetes. Cardiovasc Res 85, 473-483
48. Ayala-Torres, S., Chen, Y., Svoboda, T., Rosenblatt, J., and Van Houten, B. (2000) Analysis of gene-specific DNA damage and repair using quantitative polymerase chain reaction. Methods 22, 135-147
49. Gonzalez-Hunt, C. P., Rooney, J. P., Ryde, I. T., Anbalagan, C., Joglekar, R., and Meyer, J. N. (2016) PCR-based analysis of mitochondrial DNA copy number, mitochondrial DNA damage, and nuclear DNA damage. Curr Protoc Toxicol 67, 20 11 21-20 11 25
50. Das, A. K., Das, J. P., and Chandrasekar, S. (1987) Specific heart muscle disease in diabetes mellitus--a functional structural correlation. Int J Cardiol 17, 299-302
51. Sharma, V. S., Pilz, R. B., Boss, G. B., and Magde, D. (2003) Reactions of nitric oxide with vitamin B12 and its precursor, cobinamide. Biochemistry 42, 8900-8908
52. Broderick, K. E., Singh, V., Zhuang, S., Kambo, A., Chen, J. C., Sharma, V. S., Pilz, R. B., and Boss, G. R. (2005) Nitric oxide scavenging by the cobalamin precursor cobinamide. J. Biol. Chem 280, 8678-8685
53. Broderick, K. E., Feala, J., McCulloch, A., Paternostro, G., Sharma, V. S., Pilz, R. B., and Boss, G. R. (2006) The nitric oxide scavenger cobinamide profoundly improves survival in a Drosophila Melanogaster model of bacterial sepsis. FASEB J 20, 1865-1873
54. Reboucas, J. S., Spasojevic, I., and Batinic-Haberle, I. (2008) Pure manganese (III) 5,10,15,20-tetrakis(4-benzoic acid) porphyrin (MnTBAP) is not a superoxide dismutase mimic in aqueous systems: a case of structure-activity relationship as a watchdog mechanism in experimental therapeutics and biology. J Biol Inorg Chem 13, 289-302
55. Tickoo, S., and Russell, S. (2002) Drosophila melanogaster as a model system for drug discovery and pathway screening. Curr. Opin. Pharmacol 2, 555-560
56. Schleicher, R. L., Carroll, M. D., Ford, E. S., and Lacher, D. A. (2009) Serum vitamin C and the prevalence of vitamin C deficiency in the United States: 2003-2004 National Health and Nutrition Examination Survey (NHANES). Am J Clin Nutr 90, 1252-1263
57. Stabler, S. P., Marcell, P. D., Podell, E. R., and Allen, R. H. (1987) Quantitation of total homocysteine, total cysteine, and methionine in normal serum and urine using capillary gas chromatography-mass spectrometry. Anal Biochem 162, 185-196
58. Forman, H. J., Zhang, H., and Rinna, A. (2009) Glutathione: overview of its protective roles, measurement, and biosynthesis. Mol Aspects Med 30, 1-12
59. Li, Z., Gherasim, C., Lesniak, N. A., and Banerjee, R. (2014) Glutathione-dependent one-electron transfer reactions catalyzed by a B12 trafficking protein. J Biol Chem 289, 16487-16497
60. Wong, H. S., Dighe, P. A., Mezera, V., Monternier, P. A., and Brand, M. D. (2017) Production of superoxide and hydrogen peroxide from specific mitochondrial sites under different bioenergetic conditions. J Biol Chem 292, 16804-16809
61. Steven, S., Frenis, K., Oelze, M., Kalinovic, S., Kuntic, M., Bayo Jimenez, M. T., Vujacic-Mirski, K., Helmstadter, J., Kroller-Schon, S., Munzel, T., and Daiber, A. (2019) Vascular inflammation and oxidative stress: major triggers for cardiovascular disease. Oxid Med Cell Longev 2019, 7092151
62. Petrovic, S., Arsic, A., Ristic-Medic, D., Cvetkovic, Z., and Vucic, V. (2020) Lipid peroxidation and antioxidant supplementation in neurodegenerative diseases: a review of human studies. Antioxidants (Basel) 9
63. Cammisotto, V., Nocella, C., Bartimoccia, S., Sanguigni, V., Francomano, D., Sciarretta, S., Pastori, D., Peruzzi, M., Cavarretta, E., D'Amico, A., Castellani, V., Frati, G., Carnevale, R., and Group, S. (2021) The role of antioxidants supplementation in clinical practice: focus on cardiovascular risk factors. Antioxidants (Basel) 10
64. Forman, H. J., and Zhang, H. (2021) Targeting oxidative stress in disease: promise and limitations of antioxidant therapy. Nat Rev Drug Discov 20, 689-709
65. Ali, S. S., Hardt, J. I., Quick, K. L., Kim-Han, J. S., Erlanger, B. F., Huang, T. T., Epstein, C. J., and Dugan, L. L. (2004) A biologically effective fullerene (C60) derivative with superoxide dismutase mimetic properties. Free Radic Biol Med 37, 1191-1202
66. Kelley, E. E., Khoo, N. K., Hundley, N. J., Malik, U. Z., Freeman, B. A., and Tarpey, M. M. (2010) Hydrogen peroxide is the major oxidant product of xanthine oxidase. Free Radic Biol Med 48, 493-498
67. Kalyanaraman, B., Darley-Usmar, V., Davies, K. J., Dennery, P. A., Forman, H. J., Grisham, M. B., Mann, G. E., Moore, K., Roberts, L. J., 2nd, and Ischiropoulos, H. (2012) Measuring reactive oxygen and nitrogen species with fluorescent probes: challenges and limitations. Free Radic Biol Med 52, 1-6
68. Zielonka, J., Vasquez-Vivar, J., and Kalyanaraman, B. (2008) Detection of 2-hydroxyethidium in cellular systems: a unique marker product of superoxide and hydroethidine. Nat Protoc 3, 8-21
69. Chaudhry, Z. Z., Morris, D. L., Moss, D. R., Sims, E. K., Chiong, Y., Kono, T., and Evans-Molina, C. (2013) Streptozotocin is equally diabetogenic whether administered to fed or fasted mice. Lab Anim 47, 257-265
70. Butler, J., Jayson, G. G., and Swallow, A. J. (1975) The reaction between the superoxide anion radical and cytochrome c. Biochim Biophys Acta 408, 215-222
71. M. Wolak, G. Stochel, M. Hamza, R. Eldik, Aquocobalamin (vitamin B12a) does not bind NO in aqueous solution. Nitrite impurities account for observed reaction. Inorg Chem 39, 2018-2019 (2000)
72. H. J. Forman, I. Fridovich, Superoxide dismutase: a comparison of rate constants. Arch Biochem Biophys 158, 396-400 (1973)
73. J. J. Perry et al., Contribution of human manganese superoxide dismutase tyrosine 34 to structure and catalysis. Biochemistry 48, 3417-3421 (2009).

What is claimed is:

1. A method of treating oxidative stress in a subject with diabetes, the method comprising administering to a subject in need thereof an effective amount of cobinamide, a cobinamide derivative, or a salt thereof.

2. The method of claim 1, wherein the diabetes is associated with oxidative stress.

3. The method of claim 1, wherein a cobalt atom of the cobinamide, cobinamide derivative, or salt thereof independently may be coordinated with one or more ligands.

4. The method of claim 1, wherein the cobinamide, cobinamide derivative, or salt thereof is administered orally.

5. A method of treating oxidative stress in a subject, the method comprising administering to a subject in need thereof an effective amount of cobinamide, a cobinamide derivative, or a salt thereof.

6. The method of claim 5, wherein the oxidative stress contributes to diabetes-associated conditions including cardiac fibrosis, or to aortic aneurysm, lipid or protein oxidation, or DNA damage.

7. The method of claim 5, wherein a cobalt atom of the cobinamide, cobinamide derivative, or salt thereof independently may be coordinated with one or more ligands.

8. The method of claim 5, wherein the cobinamide, cobinamide derivative, or salt thereof is administered orally.

9. A method of treating a condition associated with diabetes, the method comprising administering to a subject in need an effective amount of a composition comprising an anti-oxidant to lower reactive oxygen species.

10. The method of claim 9, wherein the diabetes is associated with oxidative stress.

11. The method of claim 9, wherein the anti-oxidant is cobinamide, a combinamide derivative, or a salt thereof.

12. The method of claim 11, wherein a cobalt atom of the cobinamide, cobinamide derivative, or salt thereof independently may be coordinated with one or more ligands.

13. The method of claim 11, wherein the cobinamide, cobinamide derivative, or salt thereof is administered orally.
